(19) United States
(12) Patent Application Publication
(10) Pub. No.: US 2026/0245721 A1
(43) Pub. Date: Aug. 20, 2026

(54) DIGITAL HEALTH SYSTEMS WITH MEDICATION AGENTS

(71) Applicant: Paul Atkinson, Poway, CA (US)

(72) Inventor: Paul Atkinson, Poway, CA (US)

(21) Appl. No.: 19/356,758

(22) Filed: Oct. 13, 2025

Related U.S. Application Data
(60) Provisional application No. 63/706,106, filed on Oct. 11, 2024.

(51) Int. Cl. G16H 40/67; G16H 20/13

(52) U.S. Cl. CPC G16H 40/67; G16H 20/13

(57) ABSTRACT

Briefly, a digital health system is disclosed that comprises of one or more patient condition monitors, medication agents, a computing device and software application, and in some cases, further comprising medication delivery systems. Together they enable patients, caregivers, healthcare professionals etc. and digital healthcare systems to assess, and subsequently take actions that take into consideration the condition or state of a medication (e.g., the fitness-for-use).

### RELATED APPLICATIONS

This application claims priority to U.S. provisional application 63/706,106 filed Oct. 11, 2024 and entitled Digital Health Platforms with Medication Agents.

This application is related to U.S. Pat. No. 11,394,547 filed Aug. 4, 2016 and patent pending Ser. No. 17/734,364, both entitled Transaction Agents and Systems; U.S. Pat. No. 10,853,902 filed Oct. 2, 2020 and U.S. Pat. No. 12,026,265 filed Mar. 23, 2017, both entitled Agents and Systems for Rights Management and patent pending Ser. No. 18/760,796 filed Jul. 1, 2024, also entitled Agents and Systems for Rights Management.

This application is also related to U.S. patents pending: Ser. No. 18/119,178 filed Mar. 8, 2023 and entitled Autonomous Agents Using Smart Contracts To Enable Pay-For-Performance; Ser. No. 18/610,508 filed Mar. 20, 2023, and entitled Ad hoc Power Enabled Fail-Safe Systems; Ser. No. 18/213,770 filed Jun. 23, 2023, and entitled Hybrid Wireless Communications; Ser. No. 17/890,131 filed Feb. 4, 2024, and entitled Ad hoc Power Enabled Communication; Ser. No. 18/431,999 filed Feb. 24, 2024, and entitled Ad hoc Power Enabled Sensing; Ser. No. 18/742,852 filed Jun. 13, 2024, and entitled Ad hoc Power Enabled Sensing Devices.

This application is also related to 63/556,465 filed Feb. 22, 2024, and entitled Intelligent Agents and Systems for Temperature Sensitive Items.

Each of the patents, applications and provisional applications listed above, and elsewhere herein, are included herein by reference.

### FIELD OF THE INVENTION

The field of invention includes novel systems and methods for monitoring and managing the health of people (or animals) according to sensed conditions or states of individuals and those of the medications (and other medicinal products) they're taking, and when and where—and under what conditions or circumstances, they take them. Of particular interest are situations where an individual (a “patient”) is taking multiple medications, the conditions of each (e.g., their fitness-for-use) alone or in combination, may (negatively, or positively) affect their well being.

### BACKGROUND OF THE INVENTION

The healthcare of millions of people depends on the condition (or state) of the medications they take. For example, their fitness-for-use: the capacity to perform the purpose for which they were prescribed, may degrade over time, exposure to ambient conditions and the like. The condition of medications is often not be visually determinable and reductions in their fitness-for-use go unobserved. As do the negative consequences. In instances when the negative consequences are observed they are often unattributed to the cause—the condition of the medication. Note that hereafter, the terms ‘condition’ and ‘state’ as used in herein are generally interchangeable unless indicated otherwise by the context of their use.

The consequences ripple through healthcare systems, including automated patient sensing and monitoring systems, and-medication dispensing systems that don't account for the condition of medications they dispense.

### SUMMARY OF THE PRESENT INVENTION

A digital health system is provided that comprises:
at least one patient condition monitor for sensing/monitoring a condition or state, or change thereof, of a patient to which the PCM is applied, affixed, or inserted;
at least one medication agent (“med-agent”) bound to, embedded in or integrated with, a medication container or package containing a medication, and configured to:
   sense one or more conditions or states of, or related to, the medication; and
   progressively produce determinations of one or more sensed/monitored conditions that are indicative of the medication's utility (e.g., it's fitness-for-use);
a computing device” configured to communicate with the PCM and the med-agent (and the PCM and med-agent are configured to communicate with the computing device); and
a software application (“app”) stored on the computing device; wherein the app/computing device, using (or according to) conditional data (described later) received from the med-agent, alone or in combination with other data (e.g., related data received from a PCM, performs one or more actions.

The digital health system may also include a medication delivery system.

A prototypical use case—diabetes care—is used below to illustrate the inventions described herein. It is to be understood that those inventions, and the applications of them, are not limited to that use case or class of medications. The term “medication” is also to be understood to apply broadly to medicinal products where their utility (e.g., efficacy) depends at least in part, one a “condition” or state, and further where that condition is subject to change, e.g., in response to ambient conditions, handling or storage of the medication, exposure to, or the presence of, other medications or materials, liquids or gasses, age/decay etc.

Without limitation, a determination of a 1^{st} order condition of a medication may be produced (by one or more means and methods) by:
directly sensing, detecting, measuring, monitoring one or more ‘states’ of one or more properties or elements of the medication—at one or more moments in time, or
determining the medication's cumulative exposure to ambient conditions (thermal, liquid, moisture, gas, light/radiation, shock, vibration, motion etc.) over the life of them medication.

Without limitation, determinations of 2_{nd} order conditions may be produced, for example, using one or more determinations of 1ₛₜ order conditions. Exemplary 2_{nd} order determinations include those of the medication's efficacy (e.g., corresponding changes in a patients condition), fitness-for-use, toxicity (or other safety or risk factors), compatibility with other medications, life expectancy, expiry status etc.

Exemplary systems and methods for the generating 1ₛₜ and 2_{nd} order determinations of a medications condition are described in U.S. patent application Ser. No. 19/060,469 filed May 21, 2025 and U.S. provisional patent application 63/556,465 filed Feb. 22, 2024, both entitled Intelligent Agents And Systems For Condition Sensitive Items.

Determinations of 1^{st} or 2^{nd} order conditions, and combinations of them, may also reflect the characteristics or conditions of individual patients or populations sharing common conditions or characteristics (e.g., their sensitivity to medications, combinations of medications and other medicinal products, biochemistry, physiology, illness, history etc.). Further, determinations of 1^{st} or 2^{nd} order conditions of medications, or combinations of them, may be evaluated using determinations of other medications being taken (or not) and their respective conditions or the patient's condition. And the results of those evaluations can be used in evaluating/diagnosing patients and their conditions, the efficacy of their treatment etc. Advantageously in combination with determinations of a patient's actual (and evaluated) conditions, e.g. real-time or periodic sensing, monitoring, biological samples/specimens, lab work etc.)—or history.

The inventions described herein are also applicable to other use cases in and out of healthcare including those that do not involve medications, or individuals—e.g., equipment/machinery that depends the condition of lubricants, or a manufacturing process that depends on the condition of the raw materials of which it is comprised, or foods that according to their condition, depend on the periodic application or presence of gases, sprays, liquids, etc. the condition of them.

### BRIEF DESCRIPTION OF THE DRAWINGS

FIG. 1 illustrates the basic elements of an exemplary digital health system (PCM, Med-Agent, computing device/app).

FIG. 2 illustrates the basic elements of a digital health system in FIG. 1., further with an MDS.

FIG. 3 illustrates exemplary communications/interoperability between the elements of the digital health system of FIG. 1. with the addition of a second computing device/app (a smart watch).

FIG. 4 illustrates exemplary progression of stakeholders in a medication/med-agent.

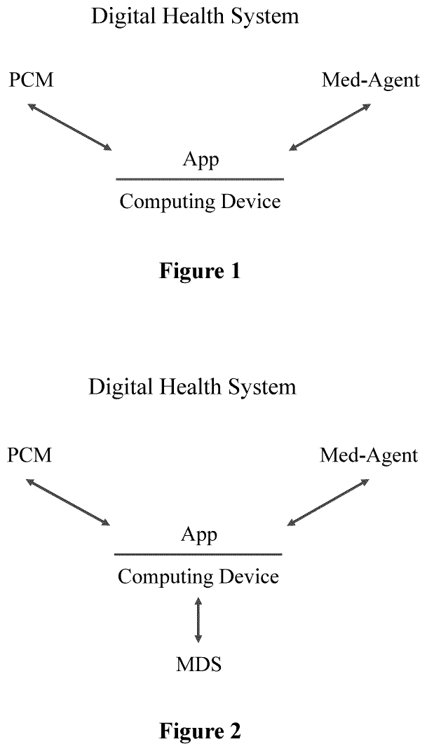

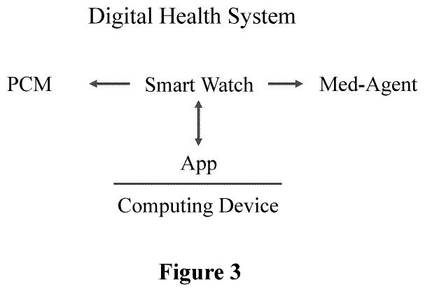

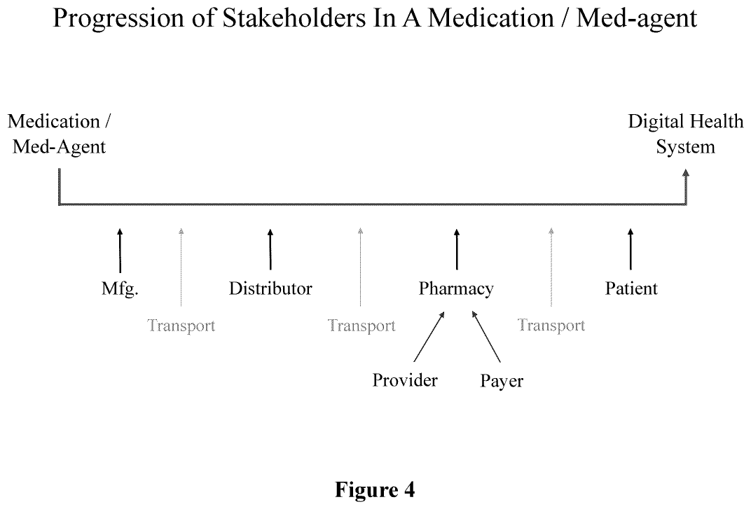

### DETAILED DESCRIPTION

Described now is a digital health system comprised of a patient condition monitor, at least one medication agent, a computing device and application. The digital health system may also include a medication delivery system.

### Patient Condition Monitors (PCMs)

Of particular interest are PCMs comprising biosensors (e.g., optical, electrochemical, enzyme-based, nucleic acid, microbial etc.) that are not physically tethered to a computing device; that is they communicate wirelessly/using wireless communication circuitry. For example, continuous glucose monitoring systems (such as Dexcom 7 or Abbott FreeStyle Libre 3 Plus). The sensors however may be of different types and the PCM may be configured to wired or wirelessly communicate with the computing device. The PCM may also be wired or electromechanically connected to, and receive power from the computing device PCMs may be single use, multi-use or reusable. They may be affixed to, inserted or embedded in, the patient and may operate for seconds, minutes, days, weeks, months.

### Medication Agents (“Med-Agents”)

Fit-for-purpose hardware “smart devices” in forms such as labels, tags, inserts, or inlays, substrates or flexible hybrid electronic devices etc. that can be attached or affixed to (inside our outside), or integrated with, individual medication containers or packages (vials, pill bottles, syringes, injectors, IV bags/bottles, ‘pods’, blister packs etc.). They may also be fabricated as part of, the containers or packages. Fit-for-purpose, med-agents operate autonomously, and at least in part, are typically powered by an internal energy store.

Advantageously, med-agents are autonomous hardware agents, AI agents, “intelligent agents” (IAs), or “progressive agents” as described in in the patents and patent applications listed in the Related Application section.

In some instances (e.g., those where the patient is taking multiple medications) some or all of the med-agents may be relatively simple, low cost devices (and fit-for-purpose passively powered), that enable a DHS to know all the medications the patient is taking, their condition and related information related to the medication itself, as well as the patient, their provenance, stakeholders, etc.

A med-agent may produce fit-for-purpose determinations (e.g., fitness-for-use, projected expiration, uncertainty/risk) or sensed and evaluated conditions (e.g., temperature, moisture/humidity, motion, light/radiation) at predetermined times, frequencies, expiration dates/times, over the useful life of a medication (or other product), or in response to changes in ambient conditions such as described in U.S. patent application Ser. No. 18/431,999 entitled Ad Hoc Power Enabled Sensing Devices.

More generally, med-agents are advantageously configured to generate and store 1^{st} and 2^{nd} order conditional information (see below). In some use cases however, they may be configured only to generate and store 1^{st} order conditional data. Med-agents are generally single use (consumable) but may also be multi-use, reusable.

### Computing Device

Stationary, transportable (e.g., an appropriately configured tablet, notebook, or terminal), or advantageously a mobile device such as a smart phone. The computing device/app may be communicatively coupled to the PCM or med-agent wirelessly (RF, optical etc.), wired, or electromechanically.

Note that PCMs and med-agents may be configured to communicate/interoperate via a computing device directly or indirectly via a secondary computing device (an app) such as a smart watch), directly with each other, or indirectly with another device (e.g., a medication delivery system such as an insulin pump or pod).

### Application (“app”)

An app (computer program) is typically the primary gateway to/from/between patients, caregivers and healthcare professionals, PCMs and med-agents, medication delivery systems, remote systems and parties (e.g., insurers, pharmacies, pharmaceutical companies, digital telehealth providers and other stakeholders—particularly when involving med-agents configured as autonomous hardware agents (and further according to, operating according to progressive smart contracts as described in the listed provisional, pending and issued patents at the end of this disclosure). The app typically resides/operates on the computing device, but may reside on a separate device communicatively coupled to the computing device.

### Conditional Data

Data (or information) regarding, related to, derived from, or dependent on, a condition or state of a medication determined by a med-agent, a use of the determination, or an outcome upon which the determination depends. Note that the term conditional data as used herein is to be understood to include conditional information as well.

### Actions

Exemplary actions performed by a computing device include (but are not limited to)*: *Actions by the computing device such as the above, are advantageously performed in-situ or with the aid of AI agents or other computing devices), and AI or other learning, analytical systems.
Analyzing and evaluating the conditional data (alone or in combination with other data/information) of one or a plurality of medications or a plurality of monitored patient conditions and correlations with patient outcomes;
   accounting/compensating for the affects of degradations in the actual fitness-for-use of each medication; and their respective contributions to patient conditions, outcomes
   further with contextual information
      comorbidity, risk factors
      dosage, dosage frequency, timing in relation to other medications or medicinal products)
      location, time
      medication distribution path/chain
      handling protocols, methods, means (e.g., use of ‘at home’ wireless refrigerators)
Making decisions, using the results of the analysis or evaluation.
Communicating conditional data, results of analyses and evaluations, actions taken and the outcomes of them (including those by patients, caregivers and healthcare professional, systems such as medication delivery systems, and to local and remote stakeholders)
   Notifications, alerts, alarms, calls-to-act (or cease-to-act)
   For remote diagnosis, telehealth, access to services
   For analysis of a medication's efficacy for a particular patient or cohort, its affect alone or in combination with other medications, on other medications and corresponding patient conditions—particularly as it relates to the condition (e.g., fitness-for-use) of each medication and other contextual variables. E.g., location, infrastructure, access to services, direct-to-patient deliver/care etc.
Adapting, adjusting or otherwise changing (temporarily or permanent) the operation of the PCM or med-agent (e.g., frequency, duration, precision, and type of sensing/ monitoring), the computing device (algorithms, data and information used to assess, evaluate, effect actions) or the operation of a medication delivery device/system (described below) if one is part of the DHS (e.g., automatic dispensing (or not) the medication, adjusting the dosage to be dispensed, communicating current status, and actions to be taken (e.g., by a patient or caregiver), alarms, alerts, notifications to local and remote parties.
Initiating/effecting transactions
   returns, replacements
   distribution of data/information, rights, costs, payments, reimbursements disbursements and other benefits (performance, outcome based etc.)

### Digital Health System (DHS)

A DHS may include more than one PCM at a time, either as single condition sensing/monitoring devices or as a single device configured to sense/monitor multiple conditions. A DHS may include more than one med-agent/medication-e.g., for a patient taking multiple medications (concurrently over time). A med-agent may also be configured to generate conditional data for a medication that is sensitive to one or more conditions (e.g., temperature, humidity or light), progressive determinations of a it's fitness-for-use.

A med-agent may be configured to passively or actively communicate current measures or determinations of a medication's current condition or state (e.g., fitness-for-use) to a computing device according to a predetermined schedule, frequency, in response to a monitored event, or on demand. Using a determination of fitness-for-use for example (alone or combination with a conditional date/information from the med-agent or a PCM) the computing device may perform an evaluation, and according to the results of the evaluation, take actions such as: notifying/alerting/alarming the patient, local or remote parties; adjust dosage and timing recommendations or the actual dosages supplied by a medication delivery system, or the operation of the PCM.

The DHS described above may further include a medication delivery system (“MDS”). Exemplary MDS's are insulin pumps or ‘pods’ that hold multiple doses of a medication (e.g., insulin), and computing devices (mobile phones, smart watches) with an app. An MDS may have its own app, it may be the one for the PCMs or one provided by third party. Any of which may support various combinations of PCMs and MDSs (and med-agents). The pumps and pods are typically attached to the patient or the patient's clothing where they are exposed to ambient conditions (e.g., temperature) that may degrade the insulin's (medication's) fitness-for-use without it being observed. Importantly, insulin in either a pump or pod may have been previously exposed to out-of-bounds conditions and the cumulative degradation in fitness-for-use (also unobserved) may have meaningfully diminished its efficacy.

When the insulin is transferred from it's container to the pump or pod, the measure of the medications fitness-for-use at that time, can be uploaded from the med-agent to the app, the pump or the pod directly (e.g., via NFC tap to connect, or using BLE). A pump or pod configured with temperature (or other condition) sensing/monitoring circuitry can then, takeover for the med-agent and continue the progressive generation of new determinations of the insulin's fitness-for-use. In this way the current determination of the insulin's fitness-for-use reflects the insulin's cumulative exposure to conditions up to the time it is actually used.

That information, alone or together with other conditional data from one or more PCMs, could be used by the computing device/app, for example, to take appropriate actions (e.g., adjust or alter the dosage to be delivered, generate notifications, alerts, alarms, or prevent the insulin from being transferred or dispensed) according to predetermined rules, instructions, conditions etc., or do so in cooperation with other systems (local or remote). The med-agent meanwhile would continue to progressively determine the fitness-for-use of the insulin remaining in the container and until it is needed and at which time the above process is would be repeated. Prior to the pump or pod being applied to dispense the insulin, the computing device/app could also alert the patient or other party (or system) as to the need to find, use a different pod or replace the insulin in the pump.

DHSs (and remote parties) via computing devices and apps, use data/information from different combinations of PCMs, med-agents and other local devices such as MDSs (and remote services), in real-time or predetermined schedules, in response to conditions or changes in them, events, local or remote queries, or actions taken. And such data information can be used by, and provide context for, AI and other systems, researchers, and other stakeholders. Among the latter are patients, caregivers, EMS services, and hospitals as well as those with financial stakes (e.g., employers, insurers, investors, inventory financiers and insurers etc.) who can efficiently risk-mitigate and price their products and services.

Systems comprising MDSs and med-agents (without or without PCMs) are not limited to medications or even healthcare. More generally it applies to systems for transferring (‘delivering’) ‘matter’ in the form of liquids, gasses, even solids (and varying states between) from one container/package/device/storage facility/system to another. E.g.,
food and beverages
industrial materials, chemicals, solvents, lubricants
cosmetics

Med-agent operations, functions, communications, actions etc. are advantageously fit-for-purpose according to embedded:
smart contracts/subcontracts and other structures
rules, rights, stakeholder directories, protocols, patient profiles, data, algorithms, computational means etc. to, for example:
   determine the fitness-for-use of the medication, dosages per sensed condition(s); ‘states’ or changes in the states of patients, context
   pair, authorize, enable communications and interoperability with other devices (e.g., computing devices, PCMs, medication delivery systems etc.)
      with or without internet access
   generate proofs of:
      conditional data (the med-agent, PCMs) its determinants and derivatives, and outcomes
      the actions/performances of the patient (caregiver and other stakeholders), med-agent, PCMs, computing device, MDS etc.
   collect and distribute conditional data and other con and proofs
   replace medications that are no longer fit-for-use, and enable distribution

DHS's may be bound to, and move with patients and as such mobile or transportable. They may also be located in locations ranging from hospitals (ERs, recovery rooms etc.), clinics, doctor's offices, residences, emergency service vehicles, ships, military bases to forward locations, urban to rural locations etc. Depending on the location the computing device may be mobile (e.g., a mobile phone or smart watch, wirelessly enabled tablet or terminal) or as in the case of an ambulance, it may be affixed to the vehicle or temporarily fixed to a gurney or wheelchair. In a hospital, it may be fixed to a ‘cart’ or ‘pole’ (e.g., an “IV pole”) which is moveable and may contain multiple, interchangeable computing/monitoring devices (and MDSs).

In some cases PCMs will be adhered or affixed to patients as previously described, while in other cases they will be temporarily bound to the patient (e.g., finger clip pulse oximetry monitors). Some PCMs will be configured to wirelessly communicate with computing devices as described previously, while others may be configured for wired connections (e.g., EKG/ECG pads). Accordingly the patient, depending on the PCM and means of communication, may be physically tethered to the computing device (and typically the app) which if stationary, restricts their mobility. Unlike as previously described, where the means of communication are wireless and the PCM goes with the patient, so can the computing device and app. MDSs are common to many of the same locations and include for example IV ‘bags’, infusion pumps and nebulizers.

Note that the location of the “sensing” circuitry and process and that of “monitoring” may be distributed fit-for-purpose between the PCM and the computing device, and if part of the DHS, an MSD. Note also that medications may be stored for extended periods of time at multiple locations, and in multiple containers before they reach the site and the time at which they are used. Thus importance of having med-agents, and med-agent enabled solutions, available even when the medication appears to be handled appropriately at any single step along the way from the point-of-manufacture to point-of-use.

It is to be understood that there are numerous embodiments of the inventions disclosed herein, and the descriptions and illustrations are exemplary.

What is claimed is:

1. A digital health system comprising:
patient condition monitor (PCM) for sensing, monitoring a condition or state of, or related to, a patient to which the PCM is affixed or inserted;
a medication agent (med-agent) bound to, embedded in or integrated with, a medication container or package containing a medication or medicinal product, and configured to:
sense one or more conditions or states of, or related to, the medication; and
progressively produce determinations of one or more sensed, monitored conditions that are indicative of the medication's utility,
a computing device configured to communicate with the PCM and the med-agent; and
a software application (app) stored on the computing device;
wherein the app, using conditional data received from the med-agent, alone or in combination with conditional data received from the PCM, performs one or more actions.

2. The digital health system of claim 1, wherein the actions are analyzing and evaluating (1) conditional data from the PCM or the med-agent, or (2) contextual information from the PCM, the med-agent, computing device or a remote party.

3. The digital health system of claim 2, wherein the actions are using the results of analyses or evaluations to make decisions.

4. The digital health system of claim 3, wherein the actions, according to the decisions, are communicating to local or remote parties conditional data or contextual information, results of analyses or evaluations, actions taken or proofs thereof.

5. The digital health system of claim 3, wherein the actions, according to the decisions, are adapting, adjusting or otherwise changing the operation of the PCM, the med-agent or the computing device.

6. The digital health system of claim 1, further comprising a medication delivery system (MDS).

7. The digital health system of claim 6, wherein the actions are analyzing or evaluating (1) conditional data from the PCM, the med-agent, or the MDS, or (2) contextual information from PCM, the med-agent, the MDS, the computing device or a remote party.

8. The digital health system of claim 7, wherein the actions are using the results of analyses or evaluations to make decisions.

9. The digital health system of claim 8, wherein the actions, according to the decisions, are communicating to local or remote parties conditional data, contextual information, results of analyses or evaluations, actions taken or proofs thereof.

10. The digital health system of claim 8, wherein the actions, according to the decisions, are adapting, adjusting or otherwise changing the operation of the PCM, the med-agent, the computing device or the MDS.

11. The digital health system of claim 1, further comprising a secondary computing device and app configured to communicate with at least two of (1) the PCM, (2) the med-agent and the (3) computing device and app.

12. The digital health system of claim 6, further comprising a secondary computing device and app configured to communicate with at least two of (1) the PCM, (2) the med-agent or(3) the computing device and app, or (4) the MDS.

13. The digital health system of claim 11, wherein the PCM and secondary device may be bound to, and move with the patient.

14. The digital health system of claim 12, wherein the PCM, MDS and secondary device may be bound to, and move with the patient.

15. The digital health system of claim 1, where the med-agent is an autonomous hardware agent, AI agent, intelligent agents, or progressive agent.

16. The digital health system of claim 7, where the med-agent is an autonomous hardware agent, AI agent, intelligent agents, or progressive agent.

17. The digital health system of claim 15, wherein the PCM or the med-agent operate according to progressive smart contracts.

18. The digital health system of claim 15, wherein the PCM or the med-agent operate according to progressive smart contracts.
